(19) United States
(12) Patent Application Publication
(10) Pub. No.: US 2025/0387584 A1
(43) Pub. Date: Dec. 25, 2025

(54) MULTI-USE AND NON-INVASIVE RESPIRATORY SYSTEM

(71) Applicant: ERADOCATE, LLC, Manchester, NH (US)

(72) Inventors: Raymond Laguerre, JR., Gardner, MA (US); Martin Johns, Mansfield, MA (US); Matthew Machata, Lake Wales, FL (US)

(21) Appl. No.: 19/248,017

(22) Filed: Jun. 24, 2025

Related U.S. Application Data
(60) Provisional application No. 63/663,570, filed on Jun. 24, 2024.
(60) Provisional application No. 63/768,366, filed on Mar. 7, 2025.

(51) Int. Cl. A61M 16/10; A61L 2/18; A61M 16/08; A61M 39/00; A61M 39/22

(52) U.S. Cl. CPC A61M 16/1075; A61L 2/18; A61M 16/0816; A61M 16/0875; A61M 39/22; A61L 2202/24; A61M 2039/0009; A61M 2202/0007; A61M 2202/0208; A61M 2205/10; A61M 2205/3334; A61M 2205/36; A61M 2240/00

(57) ABSTRACT

A multi-use, non-invasive respiratory system has a heater having at least two connection ports connectable to respiratory tubing, The heater receives a supply of oxygen. An air compressor is connected to the heater. Blended air and the oxygen exit the heater through at least one of the at least two connection ports. A disinfecting pump is connected to the heater and pumps disinfectant through the at least two connection ports. A tubing adapter system having the respiratory tubing has at least two arrangements. A first arrangement has respiratory tubing connectable to the at least two connection ports and water column for generating positive pressure is connected to at least one of the two connection ports. A second arrangement has respiratory tubing that is connectable to each of the two connection ports and is free from positive pressure generated by the water column that has an open and close adapter.

### CROSS REFERENCE TO RELATED APPLICATION

This application claims benefit of U.S. Provisional Application Ser. No. 63/663,570 filed Jun. 24, 2024 and U.S. Provisional Application Ser. No. 63/768,366 filed Mar. 7, 2025, the entire disclosures of which are incorporated herein by reference.

### FIELD OF THE DISCLOSURE

The present disclosure is generally related to respiratory systems and more particularly is related to multi-use non-invasive respiratory systems with self-disinfection.

### BACKGROUND OF THE DISCLOSURE

Low-and middle-income countries carry over 80% of the global burden of infant and childhood deaths. Respiratory failure underscores the three leading causes of under-five mortality. Low- and middle-income countries do not have the capital or personnel to manage the overwhelming burden of premature deaths of neonates and children less than five years old who die from complications of cardiopulmonary abnormalities such as respiratory distress syndrome and pneumonia. These counties are equally challenged with providing supportive measures for pediatric and adult populations. Two major factors that contribute to these ongoing respiratory challenges is that many low- and middle-income countries do not have experienced respiratory professionals who are skilled in the management of cardiopulmonary abnormalities. Additionally, low- and middle-income countries cannot afford expensive and complex respiratory equipment in the current marketplace. As a result, premature deaths, and cardiopulmonary morbidity of children five years of age or less is highest in low- and middle-income countries globally.

### SUMMARY OF THE DISCLOSURE

Embodiments of the present disclosure provide a system and method for multi-use respiratory systems with self-disinfection capabilities. Briefly described, in architecture, one embodiment of the system, among others, can be implemented as follows. A multi-use, non-invasive respiratory system has a heater having at least two connection ports. The at least two connection ports configured to connect to the respiratory tubing, wherein the heater receives a supply of oxygen. An air compressor is connected to the heater. Blended room air and the oxygen exit the heater through at least one of the at least two connection ports. A disinfecting pump is connected to the heater. The disinfecting pump is configured to pump disinfectant through the at least two connection ports. A tubing adapter system having the respiratory tubing has at least two arrangements. A first arrangement of the at least two arrangements has respiratory tubing connectable to the at least two connection ports. A water column is connected to at least one of the at least two connection ports. The water column generates a positive pressure in the respiratory tubing. A second arrangement of the at least two arrangements has respiratory tubing that is connectable to each of the at least two connection ports and is free from positive pressure generated by the water column.

The present disclosure can also be viewed as providing methods of using a multi-use, non-invasive respiratory system. In this regard, one embodiment of such a method, among others, can be broadly summarized by the following steps: Connecting respiratory tubing to a heater having at least two connection ports, wherein the heater receives a supply of oxygen; blending room air and oxygen with an air compressor connected to the heater, wherein blended room air and the oxygen exit the heater through at least one of the at least two connection ports; and connecting a tubing adapter system having the respiratory tubing in either one of a first arrangement or a second arrangement, wherein connecting the first arrangement has steps of: connecting the respiratory tubing to the at least two connection ports; connecting a water column to at least one of the two connection ports; and generating a positive pressure in the respiratory tubing with the water column, and wherein connecting the second arrangement has steps of: connecting a respiratory tubing to each of the at least two connection ports, wherein the respiratory tubing is free from positive pressure generated by the water column.

The present disclosure can also be viewed as providing methods of disinfecting respiratory tubing. In this regard, one embodiment of such a method, among others, can be broadly summarized by the following steps: Providing a disinfecting pump, the disinfecting pump having: a body; a holding chamber mounted to the body; an actuator connected to the body; and a fluid conduit having a protruding nozzle, wherein the fluid conduit is fluidly connected to the holding chamber; filling the holding chamber with a disinfectant; connecting one open end of a tubing, the tubing having two or more open ends, to the fluid conduit having the protruding nozzle, wherein the protruding nozzle at least partially extends into a portion of the tubing; closing, with a cap, the other open end of the two or more open ends of the tubing; actuating, with the actuator, the disinfecting pump, whereby actuation causes at least a portion of the disinfectant within the holding chamber to travel along the fluid pathway, into the fluid conduit, out of the protruding nozzle, and into the tubing.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a schematic diagram of a multi-use non-invasive respiratory system, in accordance with the present disclosure.

FIG. 2 provides a detailed schematic illustration of a respiratory therapy machine, in accordance with the present disclosure.

FIG. 3A illustrates a schematic view of the multi-use non-invasive respiratory system with a tubing adapter system in a first arrangement, in accordance with the present disclosure.

FIG. 3B illustrates a close up schematic view of the tubing adapter system of FIG. 3A in the first arrangement, in accordance with the present disclosure.

FIG. 4A illustrates a schematic view of the multi-use non-invasive respiratory system with a tubing adapter system in a second arrangement, in accordance with the present disclosure.

FIG. 4B illustrates a close up schematic view of the tubing adapter system of FIG. 4A in the second arrangement, in accordance with the present disclosure.

FIG. 5 illustrates a schematic diagram of a disinfecting pump, in accordance with the present disclosure.

FIG. 6 is a flowchart illustration of a method of using a multi-use, non-invasive respiratory system, in accordance with the present disclosure.

FIG. 7 is a flowchart illustration of a method of connecting the first arrangement, in accordance with the present disclosure.

FIG. 8 is a flowchart illustration of a method of connecting the second arrangement, in accordance with the present disclosure.

FIG. 9 is a flowchart illustration of a method of disinfecting respiratory tubing, in accordance with the present disclosure.

### DETAILED DESCRIPTION

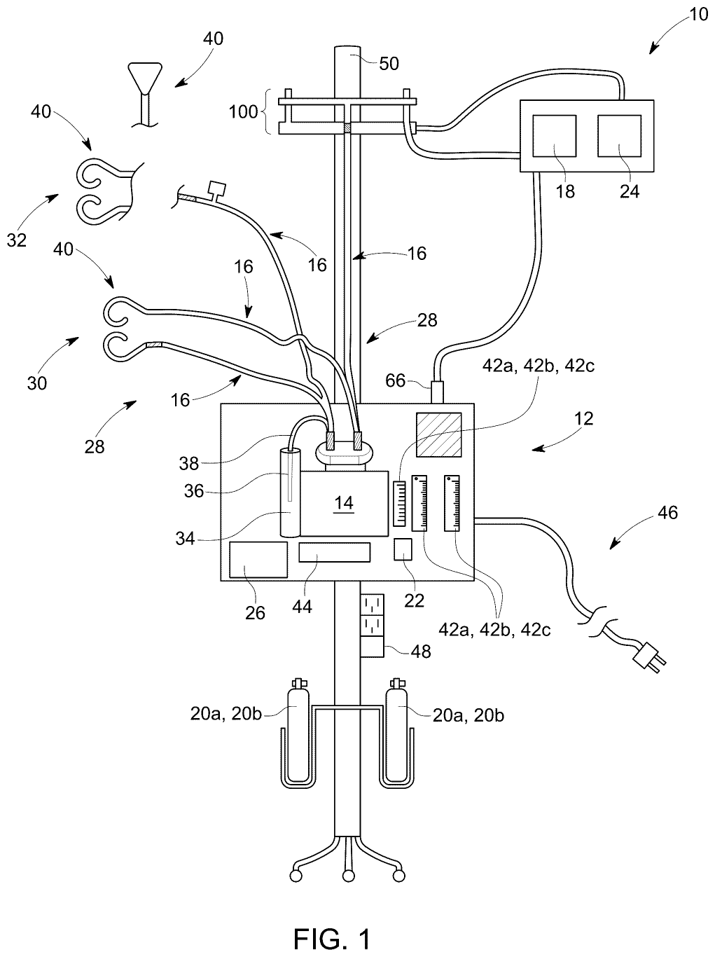

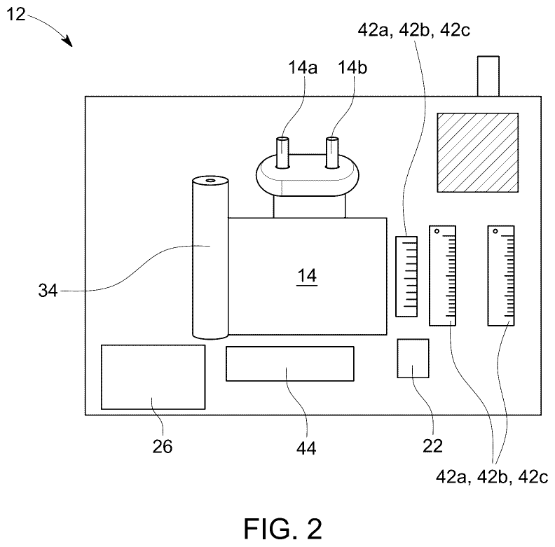

FIG. 1 is a schematic diagram of a multi-use non-invasive respiratory system 10 (hereinafter the “system 10”), in accordance with the present disclosure. FIG. 2 provides a detailed schematic illustration of a respiratory therapy machine 12, in accordance with the present disclosure. With reference to FIGS. 1-2, the system 10 has a respiratory therapy machine 12 that may provide various forms of non-invasive oxygen therapies. The respiratory therapy machine 12 may provide any one of continuous positive airway pressure (CPAP), bubble CPAP high flow oxygen, or low flow oxygen therapy to neonatal patients. The system 10 itself and the respiratory therapy machine 12 may also be configured to provide CPAP, high flow oxygen, and low flow oxygen therapy to adult and pediatric patients. The system 10 has a heater 14 which may have at least two connection ports 14a, 14b which are configured to connect to respiratory tubing 16. The heater 14 receives a supply of oxygen, which may be supplied through either wall mounted oxygen 18 or through a gas cylinder 20a containing oxygen for transport. An air compressor 22 is connected to the heater 14 and may assist in or be used to blend compressed air with oxygen. Compressed air may be supplied by a hospital source through either wall mounted air 24 or through an oxygen gas cylinder 20a containing. Air may also be supplied directly from the air compressor 22 which may take in ambient air and purify or filter it as needed. Blended air and oxygen exits the heater 14 through at least one of the at least two connection ports 14a, 14b. A disinfecting pump 26 may be connected to the heater 14. The disinfecting pump 26 is configured to pump disinfectant through at least two connection ports 14a, 14b and may provide self-disinfection capabilities to the system 10.

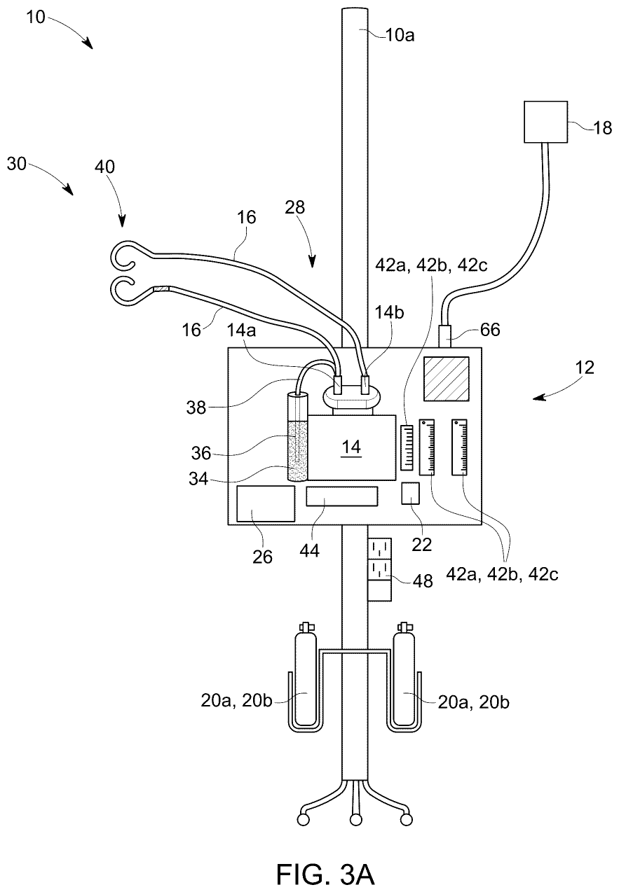

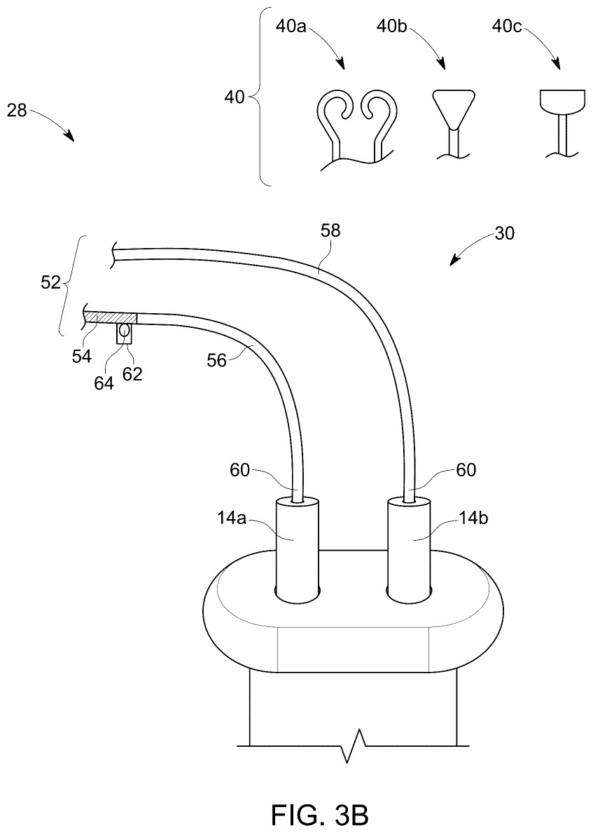

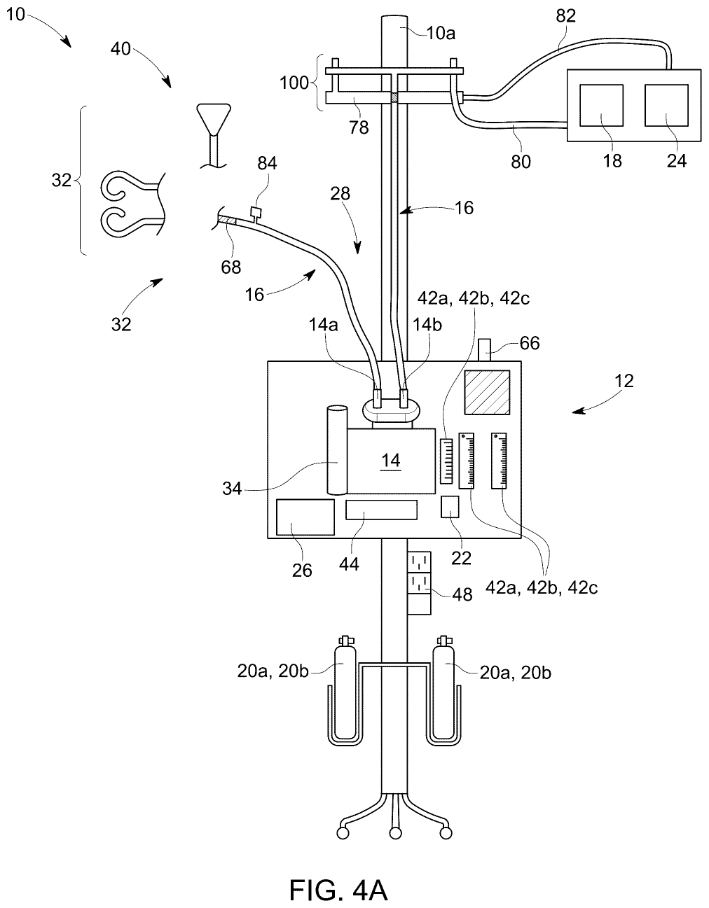

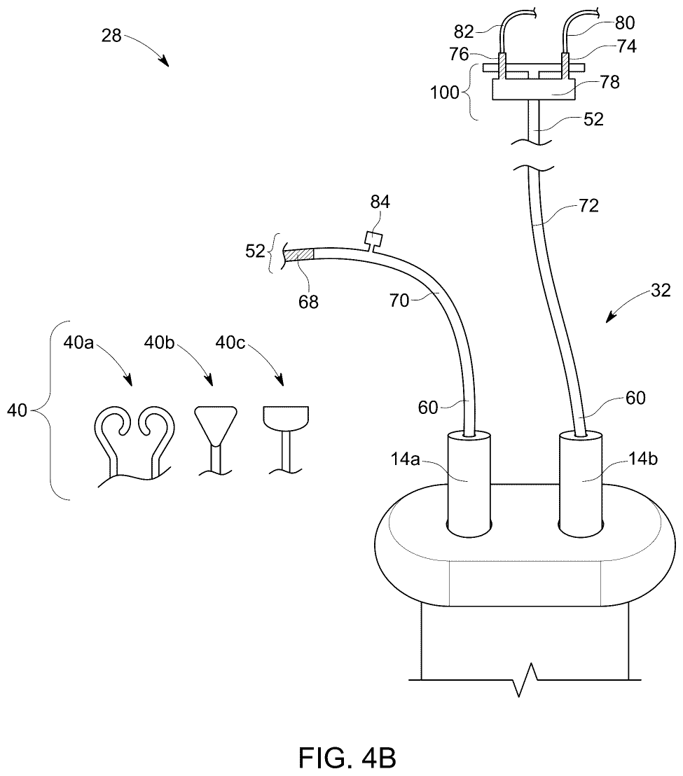

The system 10 also has a tubing adapter system 28 having the respiratory tubing 16. The tubing adapter system 28 has at least two arrangements. The first arrangement 30 has respiratory tubing 16 that is connectable to ports 14a, 14b and a fluid column or water column 34. The water column 34 is adjacent to two connection ports 14a, 14b. The water column 34 generates a positive pressure in the respiratory tubing 16 in the first arrangement 30. In the second arrangement 32 the respiratory tubing 16, when the tubing is in a certain depth in centimeters of water pressure is connectable to ports 14a, 14b and is free from positive pressure generated by the water column 34, At this time it provides high flow. The second arrangement 32 may also include a gas tube system 100 to supply medical gas, such as oxygen and air, to the respiratory therapy machine 12. Further details and aspects of the first arrangement 30 and second arrangement 32 are discussed relative to FIGS. 3A-4B and 4A-4B, respectively.

The water column 34 may be a container with a liquid such as sterile water or acetic acid. In such an example, the water column is used to generate pressure. In one example, fluid pressure may be generated when the fluid column of the water column 34 is filled with a liquid, such as acetic acid or sterile water, and when a portion of a column tubing 36, which may be the respiratory tubing 16, is submerged in the liquid. In the example of a submerged column tubing 36, the column tubing may extend out of the water column 34 to connect with at least one of the connection ports 14a, 14b through an internal tube 38. The internal tube 38 may be positioned internally within the respiratory therapy machine 12. When a portion of the column tubing 36 is submerged within the liquid of the water column 34 back pressure may be generated. The back pressure may travel through the column tubing 36, through at least one of the connection ports 14a, 14b, into the respiratory tubing 16, and to a patient wearing an air delivery device 40. The back pressure provided to the patient may aid in distending alveoli to foster gas exchange to breath. Bubbles may be formed within the liquid fluid column of the water column 34 as a patient wearing an air delivery device 40 exhales through the respiratory tubing 16. The bubbles formed within the liquid fluid column of the water column 34 may create oscillations within the lungs. These oscillations may aid in maintaining a physiologically acceptable balance of oxygen update and carbon dioxide excretion.

In the example of a water column 34 using a liquid fluid column, the column tubing 36 may be submerged at different depth to create a variety of centimeters of water pressure. When the column tubing 36 is submerged deeper in the liquid within the water column 34 using a liquid fluid column, the patient wearing an air delivery device 40 may experience greater positive pressure or positive expiratory pressure.

A respiratory therapy machine 12 with the tubing adapter system 28 arranged in the first arrangement 30 with a water column 34 having a liquid fluid column may be used in particular with neonatal patients. However, the respiratory therapy machine 12 with the tubing adapter system 28 arranged in the first arrangement 30 with water column 34 may also be arranged for use with pediatric and adult patients. Additional adjustments to the column tubing 36 depth may be made in the case of a water column 34 having liquid fluid column prior to use on pediatric and adult patients. A conventional pressure generator may also be used in lieu of a water column 34. Additional considerations and adjustments to program and adjust timing for a mechanical or electrical pressure generator may be made prior to use on a patient.

The heater 14 of the respiratory therapy machine 12 may be any heater which is conventionally used in medical equipment to heat medical gasses to a suitable temperature for inhalation. The typical temperature range for non-invasive oxygen therapy, such as CPAP, high flow oxygen, or low flow oxygen is between 31 to 37 degrees Celsius. The temperature may however be adjusted above or below this range based on the patient's need or a healthcare provider's preference. The heater 14 may be adjusted by a user or healthcare provider to increase or decrease temperature as needed for the comfort of the patient. Additionally, the heater 14 may also receive air and oxygen before sending it to the patient. As the gas passes through the heater 14, moisture and humidity is added to the gas. Humifying the heated air and oxygen allows the patient to better tolerate oxygen therapy delivered via an air delivery device 40. In particular, a patient may be able to tolerate oxygen therapy over a long period of time without discomfort or damage to the patient's airways and mucosa from dryness.

The air compressor 22 may be any air compressor that is mounted to the respiratory therapy machine 12 or is connected to the respiratory therapy machine 12. In other words, the air compressor 22 may be integral to the respiratory therapy machine 12, but may also be connected external from the respiratory therapy machine 12. The air compressor 22 may be connected to the heater 14 to supply compressed air, which may then be conditioned by the heater 14 to be supplied to the patient wearing an air delivery device 40. The air compressor 22 itself may also contain components that aid in or condition room air prior to supplying room air to the heater 14. The air compressor 22 may also be used in lieu of wall mounted hospital air 24 or gas cylinders 20b containing air to supply gas to the respiratory therapy machine 12 and therefore to a patient.

The air compressor 22 may be used to provide a pneumatic pressure to carry room air and mix with oxygen through the respiratory tubing 16 and to the air delivery device 40, forming a pneumatic system for gas delivery to a patient. The flow provided by the air compressor 22 is primarily to aid in carrying gasses from the respiratory therapy machine 12 to the air delivery device 40 via the respiratory tubing 16. In other words, the air compressor 22 provides flow with oxygen to the respiratory tubing 16 at a continuous rate to the air delivery device 40.

Air and oxygen supplied to the air delivery device 40 may be monitored using one or more flow meters 42a, 42b, 42c. The flow meters 42a, 42b, 42c may be mounted directly to the respiratory therapy machine 12. In one example, the flow meters 42 will be an oxygen flow meter 42a and air flow meter 42b. There also will be a low flow oxygen flow meter 42c. In some examples, the flow meters 42a, 42b, 42c are used to control the amount of oxygen, flow and fractional inspired oxygen (Fio2) or air that enters the respiratory therapy machine 12. In other words, oxygen and air is blended by manipulation of the flow meters to determine Fio2. The flow of oxygen or air may be controlled by a knob positioned on the respiratory therapy machine 12. The rotation of the knob either increases or decreases the flow of oxygen or room air. The low flow meter 40c works on the same principle.

While portions of the respiratory therapy machine 12 operate pneumatically, certain components such as the heater 14 and the air compressor 22 may use electricity. The heater 14 and air compressor 22 of the respiratory therapy machine 12 may be electrically powered by an AC cord 46 connected to an electrical wall outlet or a battery 44. The battery 44 may be positioned internally in the respiratory therapy machine 12, and may be made accessible for maintenance, replacement, or recharging purposes. In another example, the battery 44 may be an external battery 44 that electrically connects to an electrical connection port on the respiratory therapy machine 12 to power the heater 14 and air compressor 22. The battery 44 may be used to provide stand-alone power to the respiratory therapy machine 12 where an electrical wall outlet is not available for the AC cord 46. In one such example, the battery 44 may be used to power the respiratory therapy machine 12 during medical transport or intrahospital transport.

The battery 44 may be any conventionally used battery 44, including lithium-ion batteries, alkaline batteries, lead acid batteries, and the like. The battery 44 may be rechargeable through conventional recharging methods, including by removing the battery 44 from the respiratory therapy machine 12 to mount it on a recharging apparatus, or by a recharge circuit integrated within the respiratory therapy machine 12, such that the battery need not be removed. In one such example, the battery 44 is rechargeable by electrically connecting the AC cord 46 to an electrical wall outlet. In another example, a power strip 48 may be used to recharge the battery 44 by electrically connecting the power strip 48 to an electrical wall outlet. The power strip 48 may be configured to also output electrical power from the battery 44 to auxiliary systems and the like. Essentially, the power strip 48 may be configured as an outlet extender that can output power received from the AC cord 46 connected to an electrical wall outlet or from the battery 44.

The respiratory therapy machine 12, or at least a portion thereof, may be configured to be removably mounted to a movable pole 50. The pole 50 may be an IV pole, a pole 50 mounted to a hospital bed, or any other type of movable pole 50. The movable pole 50 allows the respiratory therapy machine 12 and the entirety of the system 10 to be easily transported. The respiratory therapy machine 12 may also be configured to be placed on a tabletop, the ground, or any flat surface. The respiratory therapy machine 12 may also be installable into a medical response vehicle such as an ambulance, medical transport vehicle, critical care transport, or air transport vehicle.

In the case of medical transport, the ability to switch between therapies and age groups may be particularly beneficial, as ambulances or other medical transport methods have limited space on board, thus carrying a dedicated system for each age group may not be feasible. The system 10 may provide benefits to hospitals in rural areas or countries with limited resources, as the system 10 may provide multi-function use, and may thus reduce the need for dedicated systems for each age group and each type of therapy.

The system 10 may be a self-cleaning system having a disinfecting pump 26 built into the respiratory therapy machine 12. In particular, various tubing 16, 38, and components of the system 10 may be disinfected using the disinfecting pump 26. Auxiliary tubing, such as tubing connected to wall mounted oxygen 18, wall mounted air 24, or a gas cylinder 20 may also be disinfected, as needed, by the disinfecting system, after being disconnected from the wall mounted oxygen 18, wall mounted air 24 or gas cylinders 20a and 20b. In some examples, the chemical substance used is a chemical substance that leaves no residue and accordingly may not require a water wash after use. In another example, the chemical used for disinfection may also be a chemical that is both food and water safe. After disinfection is complete, the system 10 may be left for a period of time to dry out. In some instances, the tubing 16, 38 and components of the system 10 may also be washed with water or other liquid subsequent to chemical disinfection treatment.

In some examples, the disinfecting pump 26 uses an electrostatic pump, ionizer, or atomizer. The use of an electrostatic pump, ionizer, or atomizer may aid in disinfection of the system 10. As charged aerosolized chemical leaves the disinfecting pump 26 and enters the tubing 16, 38 and components of the system 10, the charged aerosolized chemical may attract to the internal sidewalls of the tubing 16, 38 and components of the system 10. Therefore, a substantial portion or an entirety of the system 10 may be coated with the chemical disinfectant.

The use of a disinfecting pump 26 may substantially reduce medical waste, as tubing 16, 38 may be reused rather than discarded after individual patient use. This may provide cost benefits to facilities operating the system 10 and may also generally reduce the cost of medical care due to the reduced stockpiling of tubing 16, 38.

FIG. 3A illustrates a schematic view of the system 10 with a tubing adapter system 28 arranged in the first arrangement 30, in accordance with the present disclosure. FIG. 3B illustrates a close up schematic view of the tubing adapter system 28 of FIG. 3A arranged in the first arrangement 30, in accordance with the present disclosure. With reference to FIGS. 3A-3B, the first arrangement 30 may have particular use for neonatal and infant patients. The first arrangement 30 may be adapted for use in pediatric and adult patients, with modifications to the water column 34, as described relative to FIGS. 1-2.

The first arrangement 30 of the tubing adapter system 28 having respiratory tubing 16 is configured to provide at least one of neonatal high flow oxygen therapy, neonatal low flow oxygen therapy, or neonatal bubble CPAP to a neonatal patient in need. Neonatal high flow oxygen therapy, neonatal low flow oxygen therapy, or neonatal bubble CPAP may be provided through a variety of air delivery devices 40 connected to the respiratory tubing 16. In particular, the expiratory limb or distal end 52 of the respiratory tubing 16 may have a neonatal adapter 54 which is configured to receive at least one of a nasal cannula 40a, oxygen mask 40b, or CPAP mask 40c. The nasal cannula 40a may be adapted in size according to the flow rate of oxygen therapy. That is, in some cases, a specialized nasal cannula 40a may be desired for high flow oxygen therapy that is specific for high flow oxygen therapy, vice versa for low flow oxygen therapy. In other cases, the nasal cannula 40a used for low flow oxygen therapy may be the same as the nasal cannula 40a used for high flow oxygen therapy. In one example, the oxygen mask 40b will be a vented mask for pediatric and adult patients.

Turning now to the arrangement of the respiratory tubes 16 in the first arrangement 30, the first arrangement 30 may have a first neonatal tube 56 and a second neonatal tube 58. Each of the first neonatal tube 56 and second neonatal tube 58 have a proximal end 60, or in the case of the first arrangement 30, a proximal neonatal end, where each proximal end 60 may be connected to each connection port 14a, 14b, respectively. The first neonatal tube 56 and second neonatal tube 58 also have a distal end 52, or in the case of the first arrangement 30, a distal neonatal end. The proximal end 60 of the first neonatal tube 56 may be connected to a first connection port 14a of the two connection ports 14a, 14b. The first connection port 14a may be connected to the heater to generate humidity and to the water column34 to generate positive pressure in the first neonatal tube 56. The distal end 52 of the first neonatal tube 56 may be connectable to an air delivery device 40, which may be a neonatal air delivery device 40.

The neonatal adapter 54 may be used to form a connection between the air delivery device 40 and the distal end 52 of the first neonatal tube 56. The neonatal adapter 54 may be a universal adapter that is configured to connect to air delivery devices 40. The neonatal adapter 54 may allow connection between two or more air delivery devices 40, therefore reducing time taken to switch between different oxygen therapy types and methods for the same patient or between patients. The benefit with the neonatal adapter 54 is that one is able to switch between flow and pressure by adjusting a valve.

The second neonatal tube 58 of the respiratory tube 16 has a proximal end 60 that is connectable to a second connection port 14b of the two connection ports 14a, 14b. The second connection port 14b supplies at least one of air and oxygen or a blend of air and oxygen to the distal end 52 of the second neonatal tube 58. The distal end 52 of the second neonatal tube 58 is connectable to the delivery device 40 in the same or similar manner as the connection between the distal end 52 of the first neonatal tube 56 and the delivery device 40 and may also employ the neonatal adapter 54.

The first and second neonatal tubes 56, 58 may be made of any conventionally used medical grade respiratory tubing. In some cases, in particular, areas without immediate access to medical grade equipment and tubing, the first and second neonatal tubes 56, 58 may be any plastic, vinyl, or polyvinyl tubing that is suitable for carrying a gas heated between 31 degrees Celsius and 37 degrees Celsius.

In the first arrangement 30, the first neonatal tube 56 may be the expiratory limb or tube, and the second neonatal tube 58 may be the inspiratory limb or tube. The first neonatal tube 56, connected to the first connection port 14a and the water column 34 may generate positive pressure in the respiratory tubing 36. if the water column 34 is a liquid fluid column filled with a fluid, and a column tubing 36 is submerged into the liquid of the liquid fluid column at a predetermined depth, back pressure or positive pressure may be generated to the exhaling patient. This back pressure distends the alveoli to foster gas exchange to breath and maintains a certain physiologically acceptable residual volume in the lungs of the patient. In other words, the patient, when exhaling, overcomes the fluid pressure exerted on the submerged column tubing 36, back pressure or positive pressure is exerted on the lungs. Bubbling within the water column 34 also create oscillations within the lungs on maintain a physiologically acceptable balance of oxygen uptake and carbon dioxide excretion.

In another example, the proximal end 60 of the first neonatal tube 56 in the first arrangement 30 may be directly connected to the column tubing 36 of the water column 34. In another example, the proximal end 60 of the first neonatal tube 56 may form the column tubing 36. That is, the proximal end 60 with a Y configuration neonatal tube of the first neonatal tube 56 may be submerged in the liquid of a water column 34 at a predetermined depth to generate positive pressure or back pressure in the first neonatal tube 56.

The neonatal adapter 54 positioned on the first neonatal tube 56 may also have an adapter valve system 62 which can divert exhaled breath from the patient. When in an open state, the adapter valve system 62 allows exhaled breath to travel along the interior of the first neonatal tube 56 and into the water column 34, such that back pressure or positive pressure is generated on the lungs on the patient upon exhaling. When the adapter valve system 62 is in a closed state, an exhalation gas path along the first neonatal tube 56 is at least partially blocked, thereby restricting, reducing, or preventing exhaled gas from travelling down the first neonatal tube 56 and into the water column. When the adapter valve system 62 is in the closed state, exhaled breath may simply be vented out the distal end 52 causing high flow. This may have particular benefits when switching between bubble CPAP and low flow or neonatal high flow oxygen therapy. Instead of switching air delivery devices 40, a provider or caregiver may instead open or close the adapter valve system 62 based on the type of oxygen therapy indicated for a patient. If bubble CPAP is the desired oxygen therapy method, the adapter valve system 62 may be left in the open state. If or when bubble CPAP is no longer desired or indicated, the adapter valve system 62 may be positioned in the closed state to provide oxygen therapy without any generated positive pressure.

In an example where the neonatal adapter 54 is configured to vent exhalation gasses, the neonatal adapter 54 may be positioned anywhere along the length of the first neonatal tube 56 before the water column 34.

Oxygen and room air may be provided to the tubing adapter system 28 having the respiratory tubing 16 through the respiratory therapy machine 12. The respiratory therapy machine 12 receives oxygen and air through either the wall mounted oxygen 18, wall mounted room air (FIG. 1) or by connection to the gas cylinders having oxygen or air. Wall mounted oxygen 18 or oxygen supplied through a gas cylinder may connect to the respiratory therapy machine 12 through a respiratory therapy machine oxygen port 66. The air compressor 22 may also provide room air from ambient air. The air compressor 22 generates sufficient pneumatic pressure to push the room air, the oxygen, or the blended gas through the second neonatal tube 58, the air, oxygen, or blended gas to be received and inhaled by the patient wearing an oxygen delivery device 40.

It should be noted that the first and second neonatal tubes 56, 58 are not drawn to scale and may be sized accordingly in length, as illustrated by the break, and in diameter, based on the type of oxygen therapy desired or indicated for a patient and based on medical tubing available. The overall system 10, and in particular the first and second connection ports 14a, 14b are adapted to fit respiratory tubing 16 and other types of plastic, vinyl, or polyvinyl tubing of various sizes in diameter. FIG. 3A has several of the same or similar structures, features, and elements as described relative to FIGS. 1-2, which are not restated herein for brevity in disclosure.

FIG. 4A illustrates a schematic view of the system 10 with a tubing adapter system 28 arranged in the second arrangement 32, in accordance with the present disclosure. FIG. 4B illustrates a close up schematic view of the tubing adapter system 28 of FIG. 4A arranged in the second arrangement 32, in accordance with the present disclosure. With reference to FIGS. 4A-4B, the second arrangement 32 may have particular use for pediatric and adult patients. The second arrangement 32 of the tubing adapter system 28 having respiratory tubing 16 is configured to provide at least one of pediatric high flow oxygen therapy, pediatric low flow oxygen therapy, pediatric continuous positive airway pressure, adult low flow oxygen therapy, adult high flow oxygen therapy, or adult continuous positive airway pressure to a patient in need of oxygen therapy (collectively “patient oxygen therapy”).

Patient oxygen therapy may be provided through a variety of air delivery devices 40 connected to the respiratory tubing. In particular, the distal end 52 of the respiratory tubing in the second arrangement 32 may have a patient adapter 68 which is configured to receive at least one of a nasal cannula 40a, oxygen mask 40b, or CPAP mask 40c. The nasal cannula 40a may be adapted in size according to the flow rate of oxygen therapy. That is, in some cases, a specialized nasal cannula 40a may be desired for high flow oxygen therapy that is specific for high flow oxygen therapy, vice versa for low flow oxygen therapy. In other cases, the nasal cannula 40a used for low flow oxygen therapy may be the same as the nasal cannula 40a used for high flow oxygen therapy. The oxygen mask 40b may also be a number of oxygen masks 40b, including a non-rebreather mask, a simple oxygen mask 40b, an open oxygen mask 40b, and any other conventionally used oxygen mask 40b for oxygen therapy.

Turning now to the arrangement of the respiratory tubes 16 in the second arrangement 32. The second arrangement 32 of the respiratory tubes 16 may have a patient tube 70 that has a proximal end 60 or proximal patient end and a distal end 52 or distal patient end. The proximal end 60 of the patient tube 70 may be connectable to the first connection port 14a of the two connection ports 14a, 14b of the heater 14. The distal end 52 of the patient tube 70 may be connectable to an air delivery device 40, which may be an adult or pediatric air delivery device 40.

The patient adapter 68 may be used to form a connection between any number of air delivery devices 40 and the distal end 52 of the patient tube 70. The patient adapter 68 may be a universal adapter that is configured to connect to various air delivery devices 40 such as various nasal cannula 40a types, various oxygen mask types 40b, and CPAP masks 40c. This reduces the need for several adapters to connect various air delivery devices 40 and also reduces time between switching types of oxygen therapy for a patient. In some examples, patient adapter 68 may be able to connect to two or more air delivery devices 40 simultaneously, therefore reducing time taken to switch between different oxygen therapy types and methods for the same patent or between patients.

Turning back to the arrangement of the respiratory tubes 16 in the second arrangement 32, the gas supply tube 72 of the respiratory tubing 16 has a proximal end 60 or a proximal supply end and a distal end 52, or a distal supply end. The proximal supply end or proximal end 60 of the gas supply tube 72 may be connectable to a second connection port 14b of the at least two connection ports 14a, 14b. The distal supply end or distal end 52 of the gas supply tube 72 may be connectable to a supply of at least one of oxygen, air, or a blend of oxygen and room air. The supply supplies at least one of oxygen, room air, or a blend of oxygen and room air to the heater 14.

In particular, the distal end 52 of the gas supply tube 72 may connect to a gas tube system 100, which provides the supply of oxygen or air, and may also blend the oxygen or room air prior to entering the heater 14. It should be noted that the heater 14 may also further assist in blending oxygen with air. The gas tube system 100 has an oxygen inlet 74, an air inlet 76, and may include a connector tube 78 to initially blend or combine the oxygen and air. The oxygen inlet 74 may be configured to connect to oxygen tubing 80. The oxygen tubing 80 may also be configured to connect to wall mounted oxygen 18 or to a gas cylinder 20 containing oxygen, which provides the supply of oxygen to the gas tube system 100. The room air inlet 76 may be configured to connect to room air tubing 82. The room air tubing 82 may also be configured to connect to wall mounted air 24 or to a gas cylinder 20 containing air, which provides the supply of oxygen to the gas tube system 100. The wall mounted oxygen 18 and wall mounted air 24 may also be flowmeters, and thus, may include a knob for manipulation of the flow rate of oxygen and room air, respectively. In other words, the wall mounted oxygen 18 and wall mounted room air may be used to control the amount of oxygen or room air that enters the respiratory therapy machine 12 to determine the oxygen and air blend.

The connector tube 78 of the gas tube system 100 may also be configured to be used as a muffler to reduce the overall sound from the system 10. In such an example, the connector tube 78 may have a series of chambers and baffles positioned within an interior of the connector tube 78. The chambers and baffles of the connector tube 78 may reflect sound waves in such a way that the sound waves may interfere with one another and cancel out. This essentially reduces noise from the gas tube system by utilizing the principle of destructive interference.

The proximal end 60 of the gas supply tube 72 may be connected to the second inlet 14b of the heater 14, thereby supplying the oxygen, air, or blended gas to the heater 14 to be heated and conditioned prior to delivery to the adult or pediatric patient through the patient tube 70. In some examples, the distal end 52 of the gas supply tube 72 may also directly connect to a wall mounted oxygen 18 or to a gas cylinder 20 containing oxygen. In this example, air may be supplied to the heater 14 by the air compressor 22. The air compressor 22 may be used to provide a pneumatic pressure to carry room air, oxygen, or the blended gasses through the patient tube 70 and to the air delivery device 40, forming a pneumatic system for gas delivery to a patient.

A pressure monitor 84 may be positioned on the patient tubing 70 in the second arrangement 32. The pressure monitor 84 may be used to determine the pressure of oxygen, air, or blended gasses exerted on the patient wearing the air delivery device 40. The pressure monitor 84 may also be configured as a respiratory monitor which may sense or determine pressure in the system.

The patient tube 70 and gas supply tube 72 may be made of any conventionally used medical grade respiratory tubing. In some cases, in particular, areas without immediate access to medical grade equipment and tubing, the patient tube 70 and gas supply tube 72 may be any plastic, vinyl, or polyvinyl tubing that is suitable for carrying a gas heated between 31 degrees Celsius and 37 degrees Celsius. The oxygen tubing 80 and air tubing 82 may be made of any conventionally used medical grade respiratory tubing. In some cases, in particular, areas without immediate access to medical grade equipment and tubing, the oxygen tubing 80 and air tubing 82 may be any plastic, vinyl, or polyvinyl tubing.

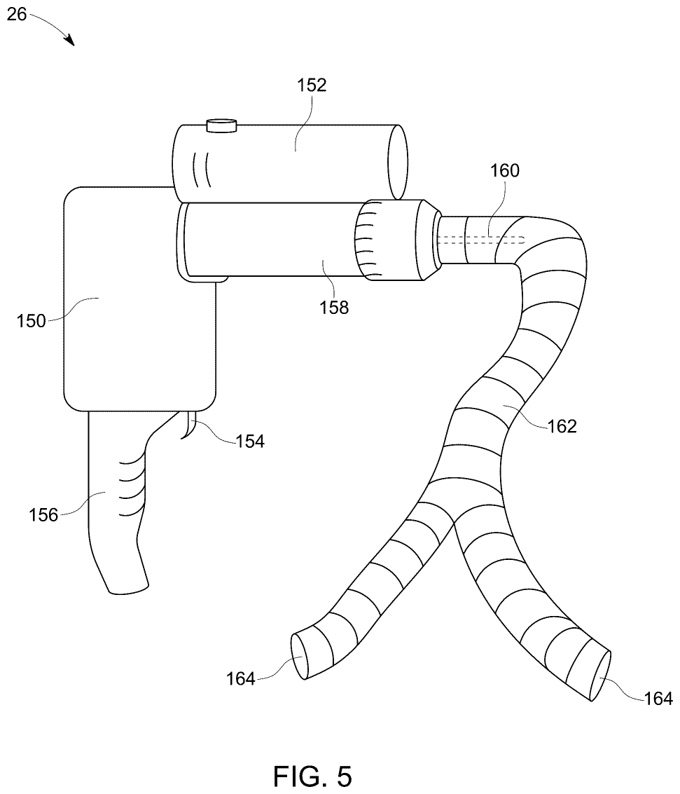

FIG. 5 illustrates a schematic diagram of a disinfecting pump 26, in accordance with the present disclosure. Provided is another example of a disinfecting pump 26 that may be a handheld device. The disinfecting pump 26 may have a housing or body 150 and a holding chamber 152 mounted to the body 150. The holding chamber 152 may be configured to receive a disinfecting chemical. Extending from the body 150 may be an actuator 154, which may be a trigger or button that activates or actuates the disinfecting pump. The body 150 of the disinfecting pump 150 may also include a handle 156 which may facilitate ease in use. A fluid conduit 158 having a protruding nozzle 160 may extend from the body 150 and may be fluidly connected to the holding chamber 152, such that upon actuation of the actuator 154, a chemical or other substance within the holding chamber 152 enters the fluid conduit 158 and exits the disinfecting pump through the protruding nozzle 160. The fluid conduit 158 may include a pump to facilitate movement of the chemical from the holding chamber 152 and out of the protruding nozzle 160. In some examples, the pump may be an electrostatic pump, ionizer, or atomizer which aerosolizes and charges the chemical exiting the protruding nozzle 160. Medical tubing 162 may be connected to the fluid conduit 158 having the protruding nozzle 160 in such a manner that the protruding nozzle 160 at least partially extends into a portion of the tubing. The medical tubing 162 may be secured to the fluid conduit 158 by a barb fitting, a push-to-fit fitting, or any other conventional methods of connecting medical tubing. The protruding nozzle 160 may aid in supplying the chemical to the medical tubing 162 by protruding into at least a portion of the tubing, which may prevent the tubing from kinking or otherwise bending where it is connected to the fluid conduit 158.

The medical tubing 162 may have multiple open ends. In the case of basic medical tubing 162, there may be only two open ends. However, certain medical tubing 162 has several open ends. Such examples include Y-tubing and the like. The other ends of the medical tubing 162 not connected to the fluid conduit 158 may be covered by a cap 164. The cap 164 may be a stopper cap, threaded cap, or the like, and may prevent fluid leakage from the medical tubing 162 during disinfection.

Use of an electrostatic pump, ionizer, or atomizer may aid in disinfection of medical tubing 162. As charged aerosolized chemical leaves the protruding nozzle 160 and enters the medical tubing 162, the charged aerosolized chemical may have an attraction to the internal sidewalls of the medical tubing 162. Therefore, a substantial portion or an entirety of the interior of the tubing 162 may be coated with the chemical disinfectant.

In some examples, the chemical substance used is a chemical substance that leaves no residue and accordingly may not require a water wash after use. In another example, the chemical used for disinfection may also be a chemical that is both food and water safe. After disinfection is complete, the medical tubing 162 may be left for a period of time to dry out. In some instances, the medical tubing 160 may also be washed with water or other liquid subsequent to chemical disinfection treatment. FIG. 4A has several of the same or similar structures, features, and elements as described relative to FIGS. 1-2, which are not restated herein for brevity in disclosure.

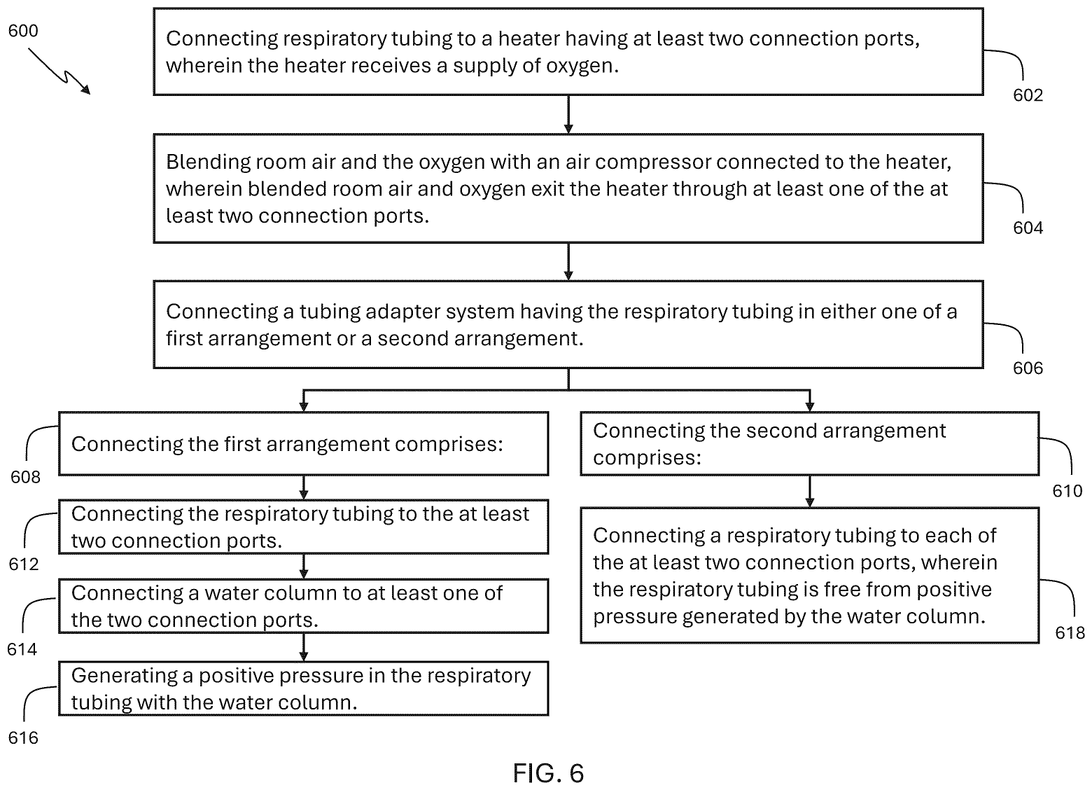

FIG. 6 is a flowchart 600 illustration of a method of using a multi-use, non-invasive respiratory system, in accordance with the present disclosure. It should be noted that any process descriptions or blocks in flow charts should be understood as representing modules, segments, portions of code, or steps that include one or more instructions for implementing specific logical functions in the process, and alternate implementations are included within the scope of the present disclosure in which functions may be executed out of order from that shown or discussed, including substantially concurrently or in reverse order, depending on the functionality involved, as would be understood by those reasonably skilled in the art of the present disclosure.

As shown in block 602, respiratory tubing is connected to a heater having at least two connection ports, wherein the heater receives a supply of oxygen. At block 604, room air and the oxygen is blended with an air compressor connected to the heater, wherein blended room air and oxygen exit the heater through at least one of the at least two connection ports. At block 606, a tubing adapter system having the respiratory tubing is connected in either one of a first arrangement or a second arrangement. The flowchart 600 splits into the first arrangement (block 608) connecting the second arrangement (block 610). Turning to connecting the first arrangement under block 608, at block 612, respiratory tubing is connected to the at least two connection ports. At block 614, a water column is connected to at least one of the at least two connection ports. At block 616, positive pressure is generated in the respiratory tubing with the water column. Turning to connecting the second arrangement under block 610, at block 618, respiratory tubing is connected to each of the at least two connection ports, wherein the respiratory tubing is free from positive pressure generated by the water column. With reference to FIGS. 1-5, it is understood that any number of additional processes, methods, and steps may be employed to arrange the system 10, its structures and elements and to use the system 10, its structures and elements.

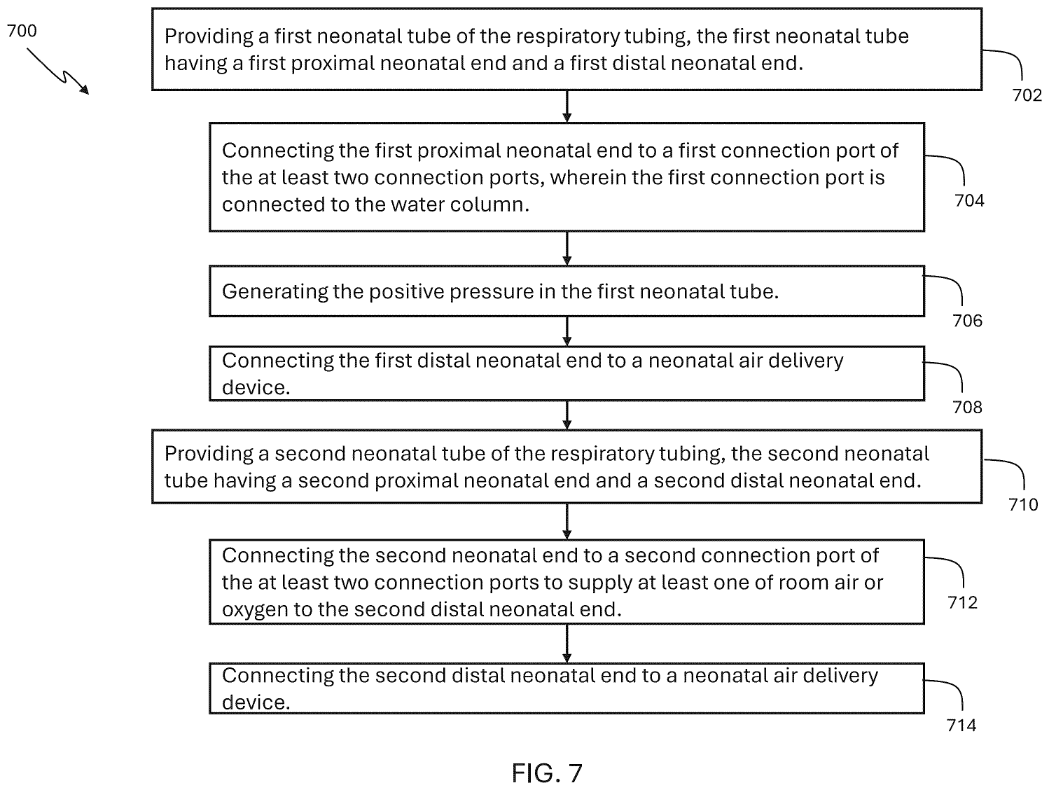

FIG. 7 is a flowchart 700 illustration of a method of connecting the first arrangement, in accordance with the present disclosure. It should be noted that any process descriptions or blocks in flow charts should be understood as representing modules, segments, portions of code, or steps that include one or more instructions for implementing specific logical functions in the process, and alternate implementations are included within the scope of the present disclosure in which functions may be executed out of order from that shown or discussed, including substantially concurrently or in reverse order, depending on the functionality involved, as would be understood by those reasonably skilled in the art of the present disclosure.

As shown in block 702, a first neonatal tube of the respiratory tubing is provided, the first neonatal tube having a first proximal neonatal end and a first distal neonatal end. At block 704, the first proximal neonatal end is connected to a first connection port of the at least two connection ports, wherein the first connection port is connected to the water column. At block 706, positive pressure is generated in the first neonatal tube. At block 708, the first distal neonatal end is connected to a neonatal air delivery device. At block 710, a second neonatal tube of the respiratory tubing is provided, where the second neonatal tube has a second proximal neonatal end and a second distal neonatal end. At block 712, the second neonatal end is connected to a second connection port of the at least two connection ports to supply at least one of room air or oxygen to the second distal neonatal end. At block 714, the second distal neonatal end is connected to a neonatal air delivery device. With reference to FIGS. 1-5, it is understood that any number of additional processes, methods, and steps may be employed to arrange the system 10, its structures and elements and to use the system 10, its structures and elements.

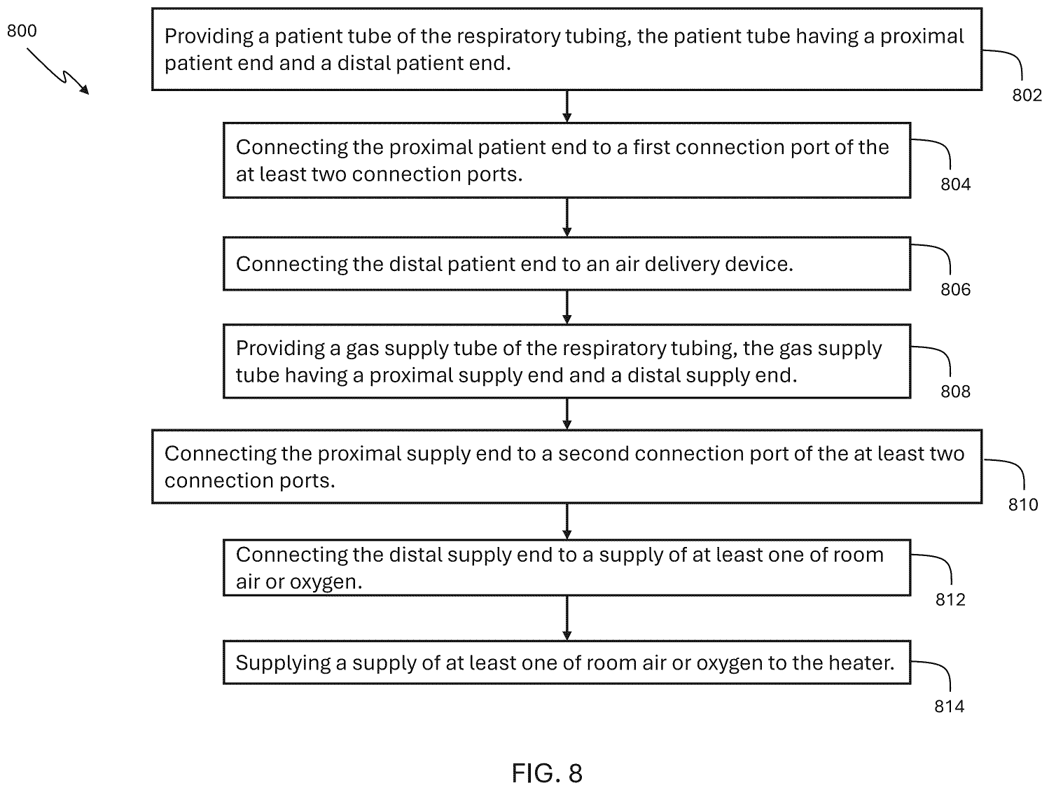

FIG. 8 is a flowchart 800 illustration of a method of connecting the second arrangement, in accordance with the present disclosure. It should be noted that any process descriptions or blocks in flow charts should be understood as representing modules, segments, portions of code, or steps that include one or more instructions for implementing specific logical functions in the process, and alternate implementations are included within the scope of the present disclosure in which functions may be executed out of order from that shown or discussed, including substantially concurrently or in reverse order, depending on the functionality involved, as would be understood by those reasonably skilled in the art of the present disclosure.

As shown in block 802, a patient tube of the respiratory tubing is provided, the patient tube having a proximal patient end and a distal patient end. At block 804, the proximal patient end is connected to a first connection port of the at least two connection ports. At block 806, the distal patient end is connected to an air delivery device. At block 808, a gas supply tube of the respiratory tubing is provided, the gas supply tube having a proximal supply end and a distal supply end. At block 810, the proximal supply end is connected to a second connection port of the at least two connection ports. At block 812, the distal supply end is connected to a supply of at least one of room air or oxygen. At block 814, a supply of at least one of room air or oxygen is supplied to the heater. With reference to FIGS. 1-5, it is understood that any number of additional processes, methods, and steps may be employed to arrange the system 10, its structures and elements and to use the system 10, its structures and elements.

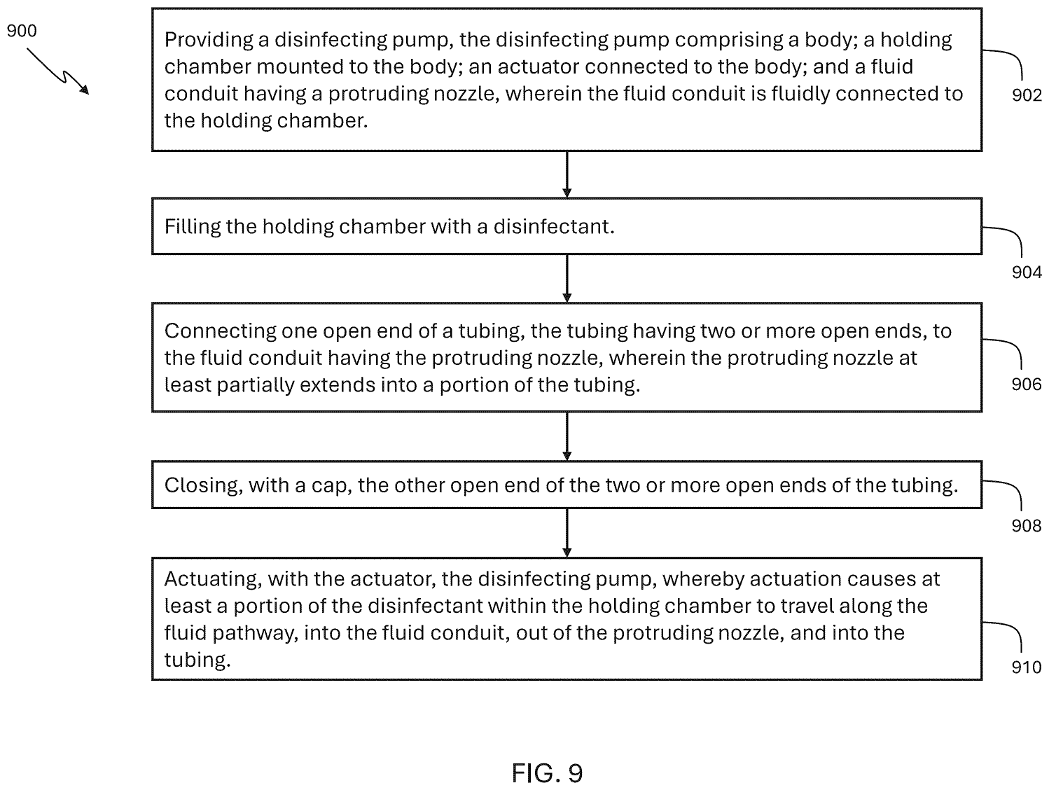

FIG. 9 is a flowchart 900 illustration of a method of disinfecting respiratory tubing, in accordance with the present disclosure. It should be noted that any process descriptions or blocks in flow charts should be understood as representing modules, segments, portions of code, or steps that include one or more instructions for implementing specific logical functions in the process, and alternate implementations are included within the scope of the present disclosure in which functions may be executed out of order from that shown or discussed, including substantially concurrently or in reverse order, depending on the functionality involved, as would be understood by those reasonably skilled in the art of the present disclosure.

As shown in block 902, A disinfecting pump is provided. The disinfecting pump has a body; a holding chamber mounted to the body; an actuator connected to the body; and a fluid conduit having a protruding nozzle, wherein the fluid conduit is fluidly connected to the holding chamber. At block 904, the holding chamber is filled with a disinfectant. At block 906, one open end of a tubing having two or more open ends is connected to the fluid conduit having the protruding nozzle, wherein the protruding nozzle at least partially extends into a portion of the tubing. At block 908 the other open end of the two or more open ends of the tubing is closed with a cap. At block 910, the disinfectant pump is actuated by an actuator, whereby actuation causes at least a portion of the disinfectant within the holding chamber to travel along the fluid pathway, into the fluid conduit, out of the protruding nozzle, and into the tubing. With reference to FIGS. 1-5, it is understood that any number of additional processes, methods, and steps may be employed to arrange the system 10, its structures and elements and to use the system 10, its structures and elements.

It should be emphasized that the above-described embodiments of the present disclosure, particularly, any “preferred” embodiments, are merely possible examples of implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of the present disclosure.

What is claimed is:

1. A multi-use, non-invasive respiratory system comprising:
a heater having at least two connection ports, the at least two connection ports configured to connect to respiratory tubing, wherein the heater receives a supply of oxygen;
an air compressor connected to the heater, wherein blended room air and the oxygen exit the heater through at least one of the at least two connection ports;
a disinfecting pump connected to the heater, the disinfecting pump configured to pump disinfectant through the at least two connection ports; and
a tubing adapter system having the respiratory tubing has at least two arrangements, wherein in a first arrangement of the at least two arrangements, the respiratory tubing is connectable to the at least two connection ports, a water column is connected to at least one of the at least two connection ports and, the water column generates a positive pressure in the respiratory tubing, wherein in a second arrangement of the at least two arrangements the respiratory tubing is connectable to each of the at least two connection ports and is free from positive pressure generated by the water column.

2. The system of claim 1, wherein the first arrangement of the tubing adapter system is configured to provide at least one of neonatal high flow oxygen therapy, neonatal low flow oxygen therapy, or neonatal bubble continuous positive airway pressure to a neonatal patient in need of oxygen therapy.

3. The system of claim 2, wherein the distal end of the respiratory tubing has a neonatal adapter configured to receive at least one of a nasal cannula, an oxygen mask, or continuous positive airway pressure mask.

4. The system of claim 1, wherein the second arrangement of the tubing adapter system is configured to provide at least one of pediatric high flow oxygen therapy, pediatric low flow oxygen therapy, pediatric continuous positive airway pressure, adult low flow oxygen therapy, adult high flow oxygen therapy, or adult continuous positive airway pressure to a patient in need of oxygen therapy.

5. The system of claim 4, wherein the distal end of the respiratory tubing has a patient adapter configured to receive at least one of a nasal cannula, an oxygen mask, or continuous positive airway pressure mask.

6. The system of claim 1, wherein the water column is connected by an internal tube to the at least one of the at least two connection ports.

7. The system of claim 1, wherein in the first arrangement:
a first neonatal tube of the respiratory tubing has a first proximal neonatal end and a first distal neonatal end, the first proximal neonatal end connectable to a first connection port of the at least two connection ports, wherein the first connection port is connected to the water column to generate the positive pressure in the first neonatal tube, wherein the first distal neonatal end is connectable to a neonatal air delivery device; and
a second neonatal tube of the respiratory tube has a second proximal neonatal end and a second distal neonatal end, the second proximal neonatal end connectable to a second connection port of the at least two connection ports to supply at least one of air or oxygen to the distal neonatal end, wherein the distal neonatal end is connectable to the neonatal air delivery device.

8. The system of claim 7, wherein the first neonatal tube of the respiratory tubing has an adapter valve system, wherein the adapter valve system is selectable between an open and closed configuration, wherein positive pressure is generated in the first neonatal tube when the adapter valve system is in the open position.

9. The system of claim 1, wherein in the second arrangement:
a patient tube of the respiratory tubing has a proximal patient end and a distal patient end, the proximal patient end connectable to a first connection port of the at least two connection ports, wherein the distal patient end is connectable to an air delivery device; and
a gas supply tube of the respiratory tubing has a proximal supply end and a distal supply end, the proximal supply end connectable to a second connection port of the at least two connection ports, wherein the distal supply end is connectable to a supply of at least one of room air or oxygen, wherein the supply supplies at least one of room air or oxygen to the heater.

10. A method of using a multi-use, non-invasive respiratory system, the method comprising:
connecting respiratory tubing to a heater having at least two connection ports, wherein the heater receives a supply of oxygen;
blending room air and oxygen with an air compressor connected to the heater, wherein blended room air and the oxygen exit the heater through at least one of the at least two connection ports; and
connecting a tubing adapter system having the respiratory tubing in either one of a first arrangement or a second arrangement, wherein connecting the first arrangement comprises:
connecting the respiratory tubing to the at least two connection ports;
connecting a water column to at least one of the two connection ports; and
generating a positive pressure in the respiratory tubing with the water column, and wherein connecting the second arrangement comprises:
connecting a respiratory tubing to each of the at least two connection ports, wherein the respiratory tubing is free from positive pressure generated by the water column.

11. The method of claim 10, further comprising disinfecting at least one of the heater, the air compressor, or the tubing adapter system with a disinfecting pump connected to the heater.

12. The method of claim 10, further comprising providing at least one of neonatal high flow oxygen therapy, neonatal low flow oxygen therapy, or neonatal bubble continuous positive airway pressure with the first arrangement of the tubing adapter system to a neonatal patient in need of oxygen therapy.

13. The method of claim 12, further comprising attaching at least one of a nasal cannula, an oxygen mask, or continuous positive airway pressure mask to a distal end of the respiratory tubing.

14. The method of claim 10, further comprising providing at least one of pediatric high flow oxygen therapy, pediatric low flow oxygen therapy, pediatric continuous positive airway pressure, adult low flow oxygen therapy, adult high flow oxygen therapy, or adult continuous positive airway pressure with the second arrangement of the tubing adapter system to a patient in need of oxygen therapy.

15. The method of claim 14, further comprising attaching at least one of a nasal cannula, an oxygen mask, or continuous positive airway pressure mask to a distal end of the respiratory tubing.

16. The method of claim 10, further comprising connecting the water column with an internal tube to at least one of the at least two connection ports.

17. The method of claim 10, wherein connecting the first arrangement comprises:
providing a first neonatal tube of the respiratory tubing, the first neonatal tube having a first proximal neonatal end and a first distal neonatal end by:
connecting the first proximal neonatal end to a first connection port of the at least two connection ports, wherein the first connection port is connected to the water column;
generating the positive pressure in the first neonatal tube; and
connecting the first distal neonatal end to a neonatal air delivery device; and
providing a second neonatal tube of the respiratory tubing, the second neonatal tube having a second proximal neonatal end and a second distal neonatal end by:
connecting the second neonatal end to a second connection port of the at least two connection ports to supply at least one of room air or oxygen to the second distal neonatal end; and
connecting the second distal neonatal end to a neonatal air delivery device.

18. The method of claim 10, wherein connecting the second arrangement comprises:
providing a patient tube of the respiratory tubing, the patient tube having a proximal patient end and a distal patient end by:
connecting the proximal patient end to a first connection port of the at least two connection ports; and
connecting the distal patient end to an air delivery device; and
providing a gas supply tube of the respiratory tubing, the gas supply tube having a proximal supply end and a distal supply end by:
connecting the proximal supply end to a second connection port of the at least two connection ports; and
connecting the distal supply end to a supply of at least one of room air or oxygen; and
supplying a supply of at least one of room air or oxygen to the heater.

19. A method of disinfecting respiratory tubing, comprising:
providing a disinfecting pump, the disinfecting pump comprising:
a body;
a holding chamber mounted to the body;
an actuator connected to the body; and
a fluid conduit having a protruding nozzle, wherein the fluid conduit is fluidly connected to the holding chamber;
filling the holding chamber with a disinfectant;
connecting one open end of a tubing, the tubing having two or more open ends, to the fluid conduit having the protruding nozzle, wherein the protruding nozzle at least partially extends into a portion of the tubing;
closing, with a cap, the other open end of the two or more open ends of the tubing; and
actuating, with the actuator, the disinfecting pump, whereby actuation causes at least a portion of the disinfectant within the holding chamber to travel along the fluid pathway, into the fluid conduit, out of the protruding nozzle, and into the tubing.

20. The method of claim 19, further comprising: applying an electric charge to at least a portion the disinfectant before entering the tubing, wherein the electric charge is applied by an electrostatic pump.
